(19) United States
(12) Patent Application Publication
(10) Pub. No.: US 2011/0263826 A1
(43) Pub. Date: Oct. 27, 2011

(54) ANTIBODIES WHICH BIND SELECTIVELY TO HAIR OF ANIMALS AND AN ANTIBODY BASED DRUG DELIVERY SYSTEM FOR ANIMALS

(75) Inventors: Stefan Hofmann, Langenfeld (DE); Hans-Juergen Hamann, Dormagen (DE); Rainer Fischer, Monschau (DE); Stefan Schillberg, Aachen (DE); Simon Oliver Vogel, Aachen (DE); Helga Schinkel, Aachen (DE)

(73) Assignee: BAYER SCHERING PHARMA AKTIENGESELLSCHAFT, Berlin (DE)

(21) Appl. No.: 12/741,036

(22) PCT Filed: Oct. 29, 2008
(86) PCT No.: PCT/EP08/09110

§ 371 Date: Jul. 12, 2011

(30) Foreign Application Priority Data
Nov. 2, 2007 (EP) EP 07021396.2

(51) Int. Cl. C07K 16/18

(52) U.S. Cl. 530/387.3; 530/389.1; 530/388.1; 530/391.9

(57) ABSTRACT

The present invention provides antibodies which bind selectively to hair of animals and an antibody based drug delivery system in which a particular formulation can be directed to animal hair. This is driven by the need of increasing the effective period of therapeutics for animal ectoparasites treatment, reducing the toxicity of these drugs and improving their release profile

The present invention provides antibodies which bind selectively to hair of animals and an antibody based drug delivery system in which a particular formulation can be directed to animal hair. This is driven by the need of increasing the effective period of therapeutics for animal ectoparasites treatment, reducing the toxicity of these drugs and improving their release profile.

Two monoclonal antibodies were selected showing high binding to the surface of dog hair. Importantly, no binding to human, horse, or cat hair was detectable. The data presented here demonstrate that the selected antibodies are suitable for designing a delivery system that directs drugs to animal hair.

### PRIOR ART

Attachment or linkage of antibodies to hair structures and beneficial use of those antibodies to link hair treatment actives or particles containing hair treatment actives has been known to the world of those skilled in the art since quite a long time. Widder (U.S. Pat. No. 3,987,161) describes a hair care product comprising an antibody containing serum which has been obtained from an animal which is capable of forming antibodies in the blood when its body is injected with an aqueous suspension of mammalian, preferably human hair. Igarashi et al. (U.S. Pat. No. 5,597,386) describes an anti-hair antibody carrying hair dye or polymer particles, e.g. polystyrene particles, containing coloring substances, thus binding the coloring actives to human hair in order to obtain benefits in hair treatment. The antibody is described as an anti-keratin antibody which has been obtained by injection of keratin polymer intramuscularly to mammals and gaining the antibody via colostrum milk, where the keratin was obtained after hydrolysis and dissolution of human hair structures. The dye-carrying particles are described as macromolecular carriers. Koyama et al. (U.S. Pat. No. 6,123,934) describes cosmetic compositions containing an antibody binding to hair and linking latex particles of a polymer or a copolymer of a vinyl monomer to the hair structure by use of this antibody. The formulations may contain cationic polymers or other ingredients to improve or repair hair structure. In this invention, the antibody has been obtained by immunizing poultry by injection of hydrolyzed human hair or particles of human hair which had been obtained by powdering whole hair fiber structures. In all of those inventions, human hair structures are used for immunization and keratin binding polyclonal antibodies have been obtained. In general, it is known that antibodies can be generated by immunization of mammals or birds with biological antigen structures which may then be used to direct beneficial actives which are linked directly or indirectly to those antibodies to the areas of need, e.g. cancer cells.

Paluzzi and co-workers (2004) have used type II keratins from cashmere as antigens to produce species-specific monoclonal antibodies. Selected antibodies were tested by two-dimensional immunoblotting for immunoreactivity with keratins isolated from cashmere and wool. Several quantitative and qualitative differences were detected enabling the specific identification of cashmere when compared to other animal fibers. However, selected monoclonal antibodies bind to all samples and only minor differences in signal intensity and protein pattern were observed between the tested species. Importantly, antibodies bind to extracted keratin type I and II proteins—binding to the hair surface has not been demonstrated.

In the veterinary field, one may benefit from those inventions by using particles containing a beneficial agent and target those agents to hair keratin structures or alternatively skin structures by using an animal hair or animal skin binding antibody. This is especially useful if one considers the option to use these particles as bodies which may release those drugs for a prolonged period of time, i.e. for weeks, month or even one year, therefore protecting the animal against pest animals, namely insects, e.g. fleas and ticks, or making use of other beneficial effects, e.g. repelling pest insects, or curing skin diseases for this long period of time. Typical formulations designed for this purpose are prepared as a one time application, showing the beneficial effect from several hours to approximately 4 weeks. Using carriers systems like polymer particles may provide the option to extend this period of time considerably. Using antibodies to link those particles to hair may provide an option to provide a linkage of the active to the hair which is sustainable enough to allow the drug carriers to stick to the hair or skin for the time period which is needed. Suitable formulations are needed. Application systems which may be useful to apply those formulations to the animal are, e.g. sprays or so called spot-on or wipe-on formulations, which bring the formulation in touch with the animal skin or fur. Actives of interest may be e.g. pyrethroids or insecticides in general, as well as systemic drugs or behavior modifying agents.

The methods described in the patents above may provide access to such drug delivery systems. However, as the antibodies described in those inventions and gained by the described methods bind to hair keratin structures, one major disadvantage appears: By applying the formulations to the animal via a spray or spot-on formulation, a strong and sustained binding to mammal hair, namely keratin structures will be obtained regardless of the species present. Therefore, the human applicator may also receive doses of actives by accidentally binding those drug carrying systems to body hair or head hair. Also, by stroking the animal, a transmission of those drug carrying actives to the human body may be possible, which might expose the pet owners to sometimes aggressive actives, e.g. pyrothroids. For application safety reasons, it would be a big advantage if one could obtain antibodies which allow to target the hair or skin of certain animal species selectively. It is also of considerable advantage to have such kind of antibody formulations available if the veterinarian has to use those formulations in stables where multiple species are kept and only one species shows the disease to be cured or only one species has to be protected from a species-specific pest.

If one uses the state-of the art methods described above to obtain antibodies to animal hair the person skilled in the art would expect to gain antibodies binding to keratin structures. Keratin is common to all mammals and protein sequences between different species are much conserved. Therefore, anti-keratin antibodies will bind to mammal hair in general.

In the present invention, surprisingly, it was found that species-specific hair binding antibodies could be obtained which bind only to species-specific animal hair surface structures, not to human hair surface or hair surface of a different species than the one whose hair had been used for immunization. Obviously, species specific antigen structures exist which can be targeted by those antibodies. A drug carrying formulation comprising those antibodies and drugs which are linked to those antibodies as molecular entity or packed into drug containing particles is able to avoid the disadvantages described above.

### DESCRIPTION OF THE EMBODIMENTS OF THE PRESENT INVENTION

The present invention provides an antibody which selectively binds to the surface of hair of animals.

Such an antibody is selected from the group comprising polyclonal antibodies, monoclonal antibodies or recombinant antibodies such as full-size antibody, dimeric secretory IgA, multimeric IgM and fragments thereof such as F(ab′)₂-fragment, Fab-fragment, Fv-fragment, single chain Fv antibody (scFv), bispecific scFv, diabody, triabody, tetrabody, dingle domain antibody (dAb), minobody or molecular recognition unit (MRU), derived from hybridoma cells, synthetic, semi-synthetic, naïve and immunocompetent phage libraries or ribosome display libraries, or by the generation of fully synthetic designer antibodies.

The antibody of the present invention selective binds to surface structures of hair of dog, cat, cattle, sheep, goat, camel, lama and/or horse.

The present invention further provides a drug delivery system to target an individual animal species, comprising the antibody, having the ability of binding selectively to the hair surfaces of said individual animal species.

Said delivery system comprising the antibody bound or attached to the surface of solid particles, thus selectively binding these particles to animal hair surface structures.

The drug delivery system of the present invention comprises of a formulation of the antibodies selectively binding to animal hair of individual species and particles containing an active ingredient, in such a way that the antibody is binding selectively to hair of said individual animal species in a first step and the particles bind to the antibody coupled to the animal hair in a second step.

Further the drug delivery system of the present invention comprises a formulation of the animal hair binding antibody linked or chemically bound to a suitable spacer/mediator/intermediate coupling agent and of particles, in such a way that the spacer/mediator/coupling agent carrying the antibody becomes selectively bound to animal hair via said antibody in a first step and that the particles become linked to the animal hair by binding to said spacer/mediator/coupling agent in a second step.

The present invention further provides a drug delivery system comprising a formulation of the animal hair binding antibody and of particles to which surface a suitable spacer/mediator/intermediate coupling agent is linked or attached, in such a way that the antibody selectively binds to animal hair in a first step and the particles carrying the spacer/mediator/coupling agent are linked to animal hair by binding to said antibodies in a second step.

The drug delivery system according to the present invention comprises the antibody selectively binding to animal hair and particles in a way that the antibody and the particles may be present in a single formulation which is applied to the animal or may be present in two different formulations (I) and (II) which becomes mixed shortly before application to the animal or, alternatively, may be present in two different formulation (I) and (II) in such a way that formulation (I) is applied to the animal in a first step and formulation (II) is applied to the animal shortly after in a second step.

The drug delivery system comprises particles which may or may not contain active ingredients or agents beneficial for the animal, namely drugs, insecticides, hair or skin caring agents, repellent agents which protect the treated animal from being attacked by harmful pest animals, e.g. insects, smell modifying agents or agents modifying the behavior of the treated animal or other non treated individual animals which interact with the animal to which the treatment is applied.

The particles may contain active ingredients or agents beneficial for the animal in a concentration suitable for its therapeutic or beneficial purpose, namely 0.01% to 99.9%.

The drug delivery system according to present invention is a system in which the particles itself contain the active ingredients or agents beneficial for the animal, namely drugs, insecticides, hair or skin caring agents, repellent agents which protect the treated animal from being attacked by harmful pest animals, smell modifying agents or agents modifying the behavior of the treated animal or other non treated individual animals which interact with the animal to which the treatment is applied.

The particles may have a diameter of 0.001 μm to 10 μm, preferably 0.1 μm to 2 μm.

The drug delivery system of the present invention, after a single treatment has been applied to the animal, allows the protection of the said animal against diseases, a continuing therapy of animal diseases or which allows the animal to benefit from the effects of said actives for a prolonged period of time, namely ranging from several hours to 365 days.

The drug delivery system of the present invention further comprises a formulation of an antibody selectively binding to animal hair and an agent beneficial to an animal in such a way that the beneficial agent is linked/coupled/bound to said antibody either directly or via a spacer/mediator/coupling agent in such a way that the linkage of said active to said antibody or spacer/mediator/coupling agent becomes broken by the environmental conditions of the animal fur or skin, thus releasing the active or part of the active or part of the active-antibody structure to perform its effect beneficial to the animal.

The drug delivery system acts in such a way that the breakage of the bonding of the active to the antibody or spacer/mediator/coupling agent happens randomly for a prolonged period of time, allowing the protection of the said animal against diseases, a continuing therapy of animal diseases or allowing the animal to benefit form the effects of said actives for a prolonged period of time, namely ranging from several hours to 365 days.

The drug delivery system as described here comprises an antibody specifically binding to animal hair or a formulation of said antibody, wherein said antibody binds to animal hair with a suitable binding strength in such a way that the amount of antibody and the amount of active ingredient attached to said antibody binding to animal hair remains large enough to maintain the desired beneficial effects for a prolonged period of time, even after the animal fur has been in contact with water for several times, e.g. by bathing or exposure to rain, or after the animal has been washed with surfactant containing solutions.

An antibody according to the present invention is able to maintain its selective binding capacity and specificity to animal hair even if it is kept at 4° C. in a solution containing water and up to 50% of organic water miscible solvents up to a period of 24 h.

The antibody is further able to maintain its selective binding capacity and specificity to animal hair even if it is formulated or kept in aqueous solutions containing other hair treatment compounds, namely surfactants, nonionic, anionic or cationic polymers and water miscible alcohols in concentrations which are typically applied in hair care formulations.

In a preferred embodiment the drug delivery system comprises the inventive antibody which is in addition bound or attached to the surface of solid particles, either directly or via a suitable spacer/mediator/intermediate coupling agent, thus selectively binding these particles to animal hair.

### DESCRIPTION OF THE FIGURES

FIG. 1 shows the binding of antibodies B7.3 and B18-4.3 on human and dog hair. Binding of the monoclonal antibodies to hair of various dog breeds and to human hair using the microtiter plate filtration ELISA with 5 ng of antibody on 1 mg hair/well. Detection with goat-anti-mouse Fc coupled to horseradish peroxidase and ABTS-substrate. All samples were analysed in three replicates and standard deviations were calculated. WHT=Westhighland terrier.

FIG. 2 is an Immunoblot of SDS-PAGE of the two antibodies B7.3 and B18-4.3. Antibody B7.3 shows the expected size for heavy and light chain, while antibody B18-4.3 has two shorter heavy chain fragments, which is caused by a mutation in the gene of the heavy chain of B18-4.3.

FIG. 3 shows a flow cytometry graph of biotinylated B18-4.3 loaded onto beads with avidin surface. Red line (second peak) shows the shift caused by goat-anti-mouse-Phycoerythrin bound to B18-4.3 indicating that the antibodies bound to the beads.

FIG. 4 shows the binding of fluorescent beads to dog hair via antibody B18-4.3. a: 0.8 μm streptavidin surface modified beads (coupled to FITC) bound via biotinylated 818-4.3 to Westhighland terrier hair. b: 1 μm Neutravidin coupled fluospheres bound via biotinylated B18-4.3 to Beagle hair, which was pretreated by washing with Tris-buffer (pH 8.8) containing 300 mM KBr at 60° C.

FIG. 5 shows the analysis of hair by confocal microscopy. Antibody B18-4.3 was primarily bound to the edges of the hair flakes. Detection by goat-anti-mouse-Alexa 488.

FIG. 6 illustrates the analysis of antigen structures recognized by antibodies B7.3 and B18-4.3. Westhighland terrier hair was washed with Tris-buffer (pH 8.8) containing 300 mM KBr and this wash fraction was concentrated by acetone precipitation. Enriched hair surface proteins (eHSP) were analysed by immunoblot. Antigens were detected by primary antibody B7.3 or B18-4.3 followed by goat-anti-mouse AP-labeled secondary antibody and staining with NBT/BCIP.

FIGS. 7a and b illustrate the binding of hair-specific antibodies B7.3 and B18-4.3 to dog, cat, horse and human hair.

FIG. 8 demonstrates the intense binding of the antibody equipped microparticles to dog hair in comparison to the poor binding to human hair.

FIG. 9 shows polystyrene microparticles with a mixture of the dog-hair specific antibodies B18-4.3 and B7.3 linked to their surfaces and containing a fluorescent dye, as they bind to beagle dog hair. The dye containing microparticles are used as binding indicators and become administered together with the same type of microparticles which contain an acaricide. This experiment was done to show the exemplary use of species-specific hair-binding antibodies and antibody equipped microparticles as controlled release drug carriers to protect dogs against ticks. The image shows the binding microparticles on some hair samples which were directly taken after the application, corresponding to 2 days after the infestation of the dog with ticks.

FIG. 10 shows the microparticles as they bind to dog hair 7 days after the application of the formulation.

FIGS. 11 a and b shows the microparticles as they bind to dog hair 14 days after the application of the formulation.

FIGS. 12 a, b and c demonstrates that even after 42 days after the administration microparticles are still visible on the dog hair proving the persistence of the antibody mediated bonding of the microparticles to dog hair.

FIG. 13 demonstrates that even after 63 days after the administration of the formulations to dog hair microparticles are still visible on the hair surface.

### GENERATION AND SELECTION OF MONOCLONAL HAIR-SPECIFIC ANTIBODIES

Animal hair-specific antibodies were generated through immunization of mice and subsequent use of hybridoma technology. Cut hair from Beagle was shredded using a homogeniser. Shredded hairs were approx. 200 μm in length.

Two mice were immunized with shredded Beagle hair. Maximal 80 μg hair sample (=20 μL shredded hair) was mixed with 40 μL GERBU adjuvant and 50 μL 1×PBS and used for immunization. Mice were immunized four times at October 11^{th}, October 25^{th}, November 3^{rd} and November 28^{th} and sacrificed at December 2^{nd}. Spleen cells were isolated and fused to myeloma cells to generate hybridomas. Limited dilution was done by pipetting cells in increasing dilutions into 96-well microotiter plates. To screen for hybridoma clones producing hair-specific antibodies a new ELISA setup was established. Partially lysed hair (10 min in Shindai reagent at RT followed by homogenisation and resuspension in carbonate buffer) was coated onto low binding 96-well microtiter plates. After addition of the hybridoma cell culture supernatant, binding of specific antibodies was detected using a secondary goat-anti mouse (GAM) antibody conjugated to horseradish peroxidase (HRPO). In total 23 polyclonal hybridoma clones (B1-23) binding to hair samples were identified. However, since partially lysed hair was coated, both antibodies binding to antigens present inside the hair and to antigens on the hair surface were selected.

To identify binders recognizing structures on the hair surface, antibodies from monoclonal hybridoma lines were tested using the Eppendorf-based ELISA. 20-50 human or animal hairs were fixed with one drop of glue to the bottom of an Eppendorf tube and unspecific binding sites were blocked with 2% (w/v) BSA in PBS. Hybridoma supernatant or 10 μg of purified monoclonal antibody was transferred to the tube and incubated for 30 minutes at room temperature. After washing specific binding was detected using a goat-anti mouse secondary antibody (labeled with HRPO) and incubation for 20 minutes at room temperature. Finally, binding was visualized using the 2,2′-azino-bis 3-ethylbenziazoline-6-sulfonic acid (ABTS) reagent as a substrate for HRPO. Colour development was measured after 30 seconds at OD 405.

Two monoclonal antibodies were identified by the Eppendorf-based ELISA. These antibodies, named B7.3 and B18-4.3, showing specific binding to the surface of animal hair were purified and characterized regarding their specificity to different hair samples.

### Purification of Monoclonal Antibodies

Monoclonal antibodies B7.3 and B18-4.3 were purified from hybridoma culture supernatant via protein G or alternatively by precipitation of ultrafiltration. Yields of purified protein ranged from 0.1 to 1.1 mg/mL. Purified antibodies were used for subsequent assays.

### Characterization of Monoclonal Antibodies

### Antibody Binding—Eppendorf-Based ELISA

To verify qualitatively the binding of purified monoclonal antibodies B7.3 and B18-4.3 to the hair surface the Eppendorf-based ELISA was used. The antibodies B7.3 and B18-4.3 bind with specificity to Yorkshire terrier hair. Importantly, no binding to human hair was detected.

Several additional Eppendorf-based ELISA were performed using the following hair samples: human, horse, cat, Schnauzer/Labrador mix, Australian shepherd, Poodle before and after washing (3 samples), Yorkshire terrier before and after washing (4 samples), Westhighland terrier before and after washing (2 samples)

A summary of the Eppendorf-based ELISAs is presented in Table 1. The data show that both monoclonal antibodies bind specifically to the surface of the hair from five canine species. No binding was observed when human, horse or cat hair was used demonstrating the strong specificity of the selected antibodies for dog hair. It seems that the monoclonal antibody B18-4.3 possesses a stronger binding activity to canine hair than B7.3. Some minor variations are present between the different hair samples. Hair treatment prior to the performance of the ELISA reduces the binding activity of all tested antibodies, although bound antibodies are still present.

TABLE 1
Results of the Eppendorf-based ELISA tests.

| | | | | | | | | | Yorkshire | | | | Westhighland | |
|---|---|---|---|---|---|---|---|---|---|---|---|---|---|---|
| | | | | Schnauzer/ | Australian | Poodle | | | Terrier | | | | Terrier | |
| Antibody | Human | Horse | Cat | Labrador | Shepherd | 1 | 2 | 3 | 1 | 2 | 3 | 4 | 1 | 2 |
| B7.3 | − | − | − | + | + | ++ * | +++ * | ++ ** | + * | ++ * | + * | + * | +++ * | +++ ** |
| B18-4.3 | − | − | − | +++ | +++ | +++ * | +++ ** | +++ ** | +++ * | +++ ** | ++ * | ++ * | +++ ** | +++ ** |

− = no binding, + = low binding (OD < 1), ++ = medium binding (OD > 1 < 3), +++ = high binding (OD > 3),
* = after washing.

### Antibody Binding and Functionality

Another type of ELISA was established to be able to compare the binding of the two antibodies B7.3 and B18-4.3 to hair quantitatively. Dog hair was cut into 2-5 mm long pieces and dissolved in PBS (5 mg/ml). 200 μl of this suspension was pipetted into each well of a 96-well microtiter filtration plate (Multiscreen HTS, membrane pore size 1.2 μm, Millipore). Buffer and substrate exchanges are done by centrifugation so that the hair in the microtiter filtration plate is not diminished in these procedures.

In preliminary experiments the maximal amount of antibody that is bound to 1 mg of hair (=well) was determined to be 5-10 ng (depending on dog race and antibody). Therefore experiments were mostly performed with 2.5 or 5 ng/well.

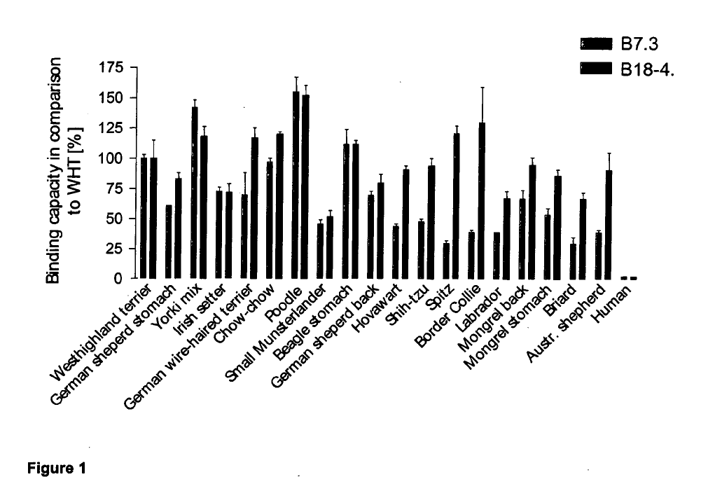

To check the binding of the two antibodies to hair of various dog races, a microtiter filtration plate ELISA was performed with hair of Westhighland terrier, German shepherd (hair from the stomach), Yorkshire terrier mix, Irish setter, German wired-hair terrier, Chow-chow, Poodle, small Munsterlander, Beagle, German shepherd (hair from the back region), Hovawart, Shih-tzu, Spitz, Border collie, Labrador, Mongrel (hair from the back), Mongrel (hair from the stomach), Briard, Australian shepherd. Also human hair was included in this test. All attained values were normalized in regard to Westhighland terrier (FIG. 1).

Binding of the purified antibodies to the hair surface was also demonstrated by immunofluorescence imaging using a confocal microscope. For example, antibody B7.3 showed strong binding to Yorkshire terrier hair. No fluorescence was observed when applying only the secondary ALEXA564 dye labeled goat-anti mouse antibody, demonstrating the specificity of B7.3 to the hair surface. Treatment of hair with acetone prior to antibody application resulted in reduced fluorescence signals. Similar results were obtained when hair samples were washed with mild detergents.

### Antibody Integrity, Stability and Functionality

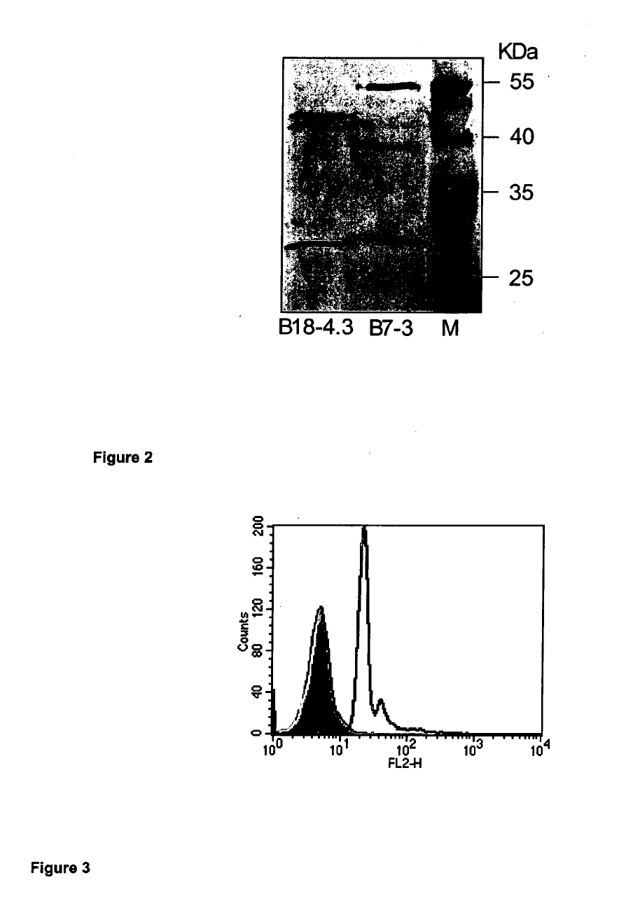

To verify the integrity of the antibody heavy and light chain, purified proteins were analysed by SDS polyacrylamide gel electrophoresis. While antibody B7.3 possesses an intact heavy and light chain of the expected size, antibody B18-4.3 has two heavy chain bands that are both smaller than the full-length heavy chain (approx 44 and 47 kDa instead of 55 kDa) (FIG. 2). One of these mutated heavy chains is a result of a frameshift mutation at the end of the C2 domain which results in a premature stop codon. The origin of the other mutated heavy chain is not yet known. No degradation products of the B7.3 and B18-4.3 heavy chains were observed demonstrating the integrity of the purified antibodies.

To analyse the stability, binding of the monoclonal antibodies was carried out in the presence of isopropanol using the Eppendorf-based ELISA. The monoclonal antibody B7.3 showed stable binding in the presence of 30% (v/v) isopropanol over a period of four months at 4° C. Binding to the canine hair was still present when the antibody was kept at 4° C. in 40% or 50% (v/v) isopropanol demonstrating the high stability of this protein. Moreover, the antibody B7.3 was stable at 22° C. in the presence of 20% (v/v) isopropanol.

Stability of B18-4.3 was even better. The monoclonal antibody showed stable binding to the hair after storage in the presence of 40% (v/v) isopropanol over a period of four months at 4° C.

In addition, stability and functionality of monoclonal antibodies was tested in formulation solution containing 6% (v/v) Luviskol VA 64 W (BASF), 5% (v/v) Luviquat PQ 11 PN (BASF), 0.3% (v/v) Pluracare E400 PEG-8 (BASF), 0.2% (v/v) Q2-5220 Resin (DOW Corning) and 15% (v/v) isopropanol. After storage for 3 days at 4° C. in the formulation antibody binding was tested using the Eppendorf-based ELISA indicating that B7.3 and B18-4.3 possess a high stability in the formulation.

### Antibody Binding Capacity

To determine the maximum binding capacity, dilutions of B18-4.3 were tested in the Eppendorf-based ELISA using a defined number of Yorkshire terrier hairs (20 hairs, each 20 mm long). The surface of the hairs in each Eppendorf tube corresponded to 3.15 mm². According to the ELISA 2.5-5.0 ng of antibody B18-4.3 binds to 3.15 mm² of hair surface.

### Biotinylation of Antibodies

To analyse the influence of binding partners on antibody functionality, purified monoclonal antibodies were biotinylated and subsequently bound to avidin beads, which was verified by FACS analysis (FIG. 3). Binding to canine hair was analysed using the Eppendorf-based ELISA. Binding of the biotinylated antibodies B7.3 and B18-4.3 was detected via avidin conjugated to HRPO demonstrating that biotinylation and binding partners do not affect antibody functionality.

### Antibody Fusion to Beads

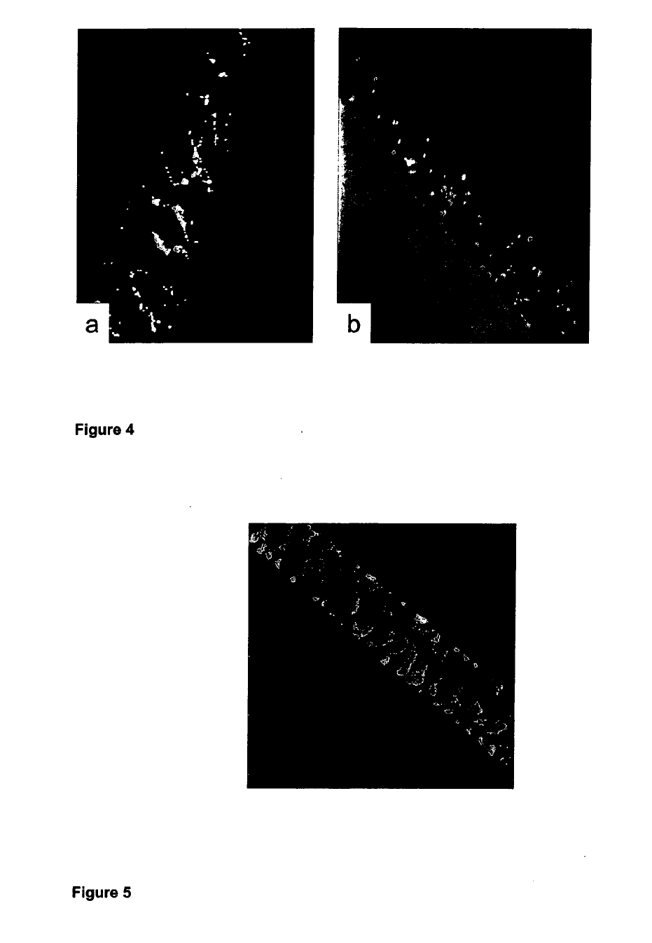

To analyse the binding of monoclonal antibody coupled to beads, hair from Westhighland terrier was pretreated with 1×PBS containing 0.5% (v/v) Tween 20 to remove contamination and not tightly fixed antigen structures. Subsequently, biotinylated antibody B18-4.3 (0.8 mg/mL) was applied and bound antibody was detected using streptavidin coated FITC fluorescent beads (Kisker). Bead size was 0.8 μm. In addition, biotinylated antibody B18-4.3 was coupled to streptavidin coated FITC fluorescent beads and the complex was applied to the hair sample to analyse binding (FIG. 4a).

Experiments show that biotinylated B18-4.3 loaded on 1 μm Neutravidin coupled fluospheres (Invitrogen) shows high binding on Beagle hair even if the hairs were pretreated by washing with Tris-buffer (pH 8.8) containing 300 mM KBr at 60° C. This indicates the high stability of the antigen on the hair surface (FIG. 4b).

In case of the 2 μm beads this effect was less pronounced which might indicate that added beads are bound by several antibodies present on the hair surface. Importantly, experiments with 10 μm beads were negative. No staining was observed because beads were lost during washing procedures.

Analysis of hair samples by confocal microscopy indicated that antibodies were primarily bound to the edges of the hair flakes (FIG. 5).

### Identification of the Antibody Binding Site

Results presented in Table 1 indicate that at least a portion of the surface antigen can be removed by washing hair samples with mild detergents. Removal of antigen structures led to reduced antibody binding. However, antibody binding is still detectable demonstrating that a significant portion of the antigen remains connected to the hair surface.

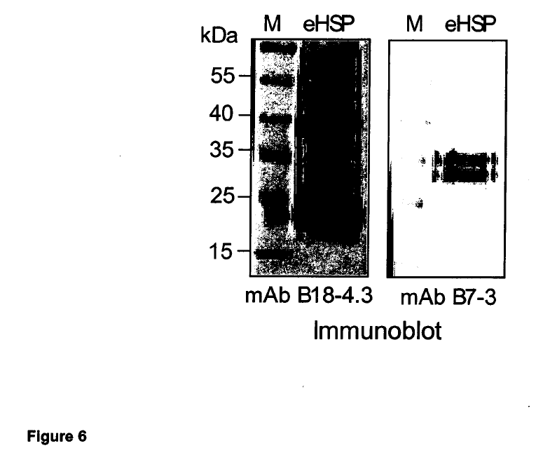

When hair of Westhighland terrier was washed with Tris-buffer (pH 8.8) containing 300 mM KBr and this wash fraction was concentrated, a preparation of enriched hair surface proteins (eHSP) was the result. These eHSP were analysed by SDS-PAGE and immunoblot, using either of the two antibodies B7.3 or B18-4.3. The result shows that B7.3 binds to structures that have a size of 32 KDa and 38 KDa while B18-4.3 detects three bands at 21 KDa, 28 KDa and 40 KDa (FIG. 6). Therefore, the two antibodies have a different specificity.

### Cloning of Variable Antibody Chains

To secure the genetic information of the monoclonal antibodies, the variable antibody domains which determine the antibody binding specificity were cloned. The following steps were performed:
RNA isolation from the hybridoma clones
Reverse transcription into cDNA
Amplification of variable regions from antibody heavy and light chain by PCR
Fusion of both variable domains through a short linker fragment by splice overlap extension (SOE) PCR
Cloning of the single chain antibody (scFv) fragment into a bacterial vector
Transformation of bacteria
Plasmid isolation and sequencing

The integrity of scFvB7.3 and scFvB18-4 genes has been confirmed by sequencing.

ScFvB18-4 was tested for its binding to dog hair. Binding of scFvB18-4 alone or coupled to beads via biotin-avidin to hair samples was demonstrated by ELISA.

### Binding of Hair-Specific Antibodies B7.3 and B18-4.3 to Dog, Cat, Horse and Human Hair

Each hair sample was measured three times by ELISA. The diagrams give mean and standard deviation of the samples. Two ELISAs were run in parallel and each diagram is the result of one ELISA. Where the same sample turns up in both diagrams (Westhighland terrier, European housecat), one hair sample was used to pipette both ELISA plates.

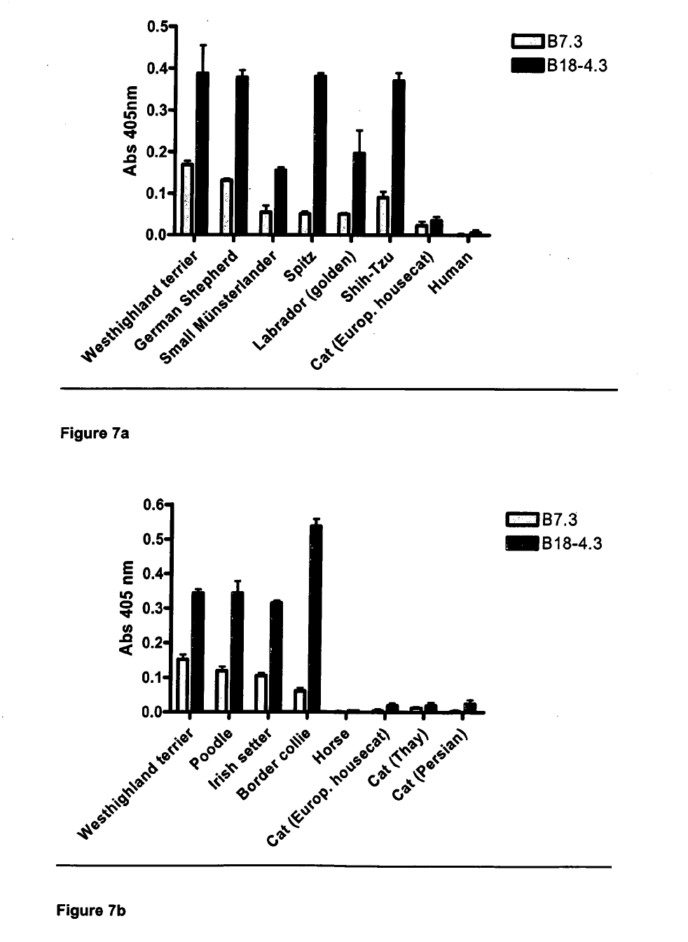

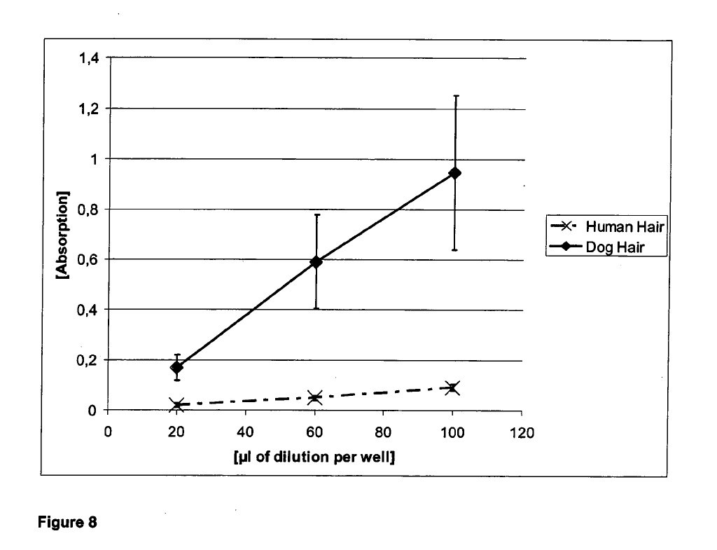

1 mg of hair was pippeted/well. Blocking was done with 2% (w/v) milk powder in PBS for 1 h. As a first antibody either B7.3 or B18-4.3 was used at a concentration of 50 ng/ml in PBS, where 100 μl antibody solution was used per well. Incubation was done at room temperature for 1 h. Secondary antibody was a goat-anti-mouse (anti IgG, IgM and IgA) coupled to a peroxidase; the antibody was diluted 1:5000 in PBS and 100 μl/well was used. Incubation was done at room temperature for 1 h. Finally each well was incubated with 100 μl of the substrate ABTS for 42 min. Reading of the resulting colour reaction was performed at 405 nm. Between the various steps the plates were washed with twice 200 μl PBS-T. As a control three wells of each hair sample were incubated with an unspecific mouse antibody and otherwise treated as described above. The mean of each control was subtracted from the results with B7.3 and B18-4.3 of the same hair sample (FIG. 7).

### Generation of Horse Hair-Specific Antibodies

Horse hair-specific antibodies were generated through immunization of mice and subsequent use of hybridoma technology. Hair from horse was washed with PBS-T (1×PBS, Tween20 0.05%) shredded using a homogeniser. Shredded hairs were approx. 200 μm in length.

Two mice were immunized with washed, shredded horse hair. Maximal 80 μg hair sample (=20 μL shredded hair) was mixed with 40 μL GERBU adjuvant and 50 μL 1×PBS and used for immunization. Mice were immunized six times at July 8^{th}, July 22^{th}, August 5^{th}, August 18^{th}, August 26^{th} and September 8^{th} and sacrificed at September 10^{th}. Spleen cells were isolated and fused to myeloma cells to generate hybridomas. Polyclonal hybridoma clones were screened for production of horse-hair-specific antibodies with an ELISA setup as described above. Shredded horse hair in 1×PBS was coated onto low binding 96-well microtiter plates. After addition of the hybridoma cell culture supernatant, binding of specific antibodies was detected using a secondary goat-anti mouse (GAM) antibody conjugated to horseradish peroxidase (HRPO). For detection of antibody binding to hair ABTS-substrate was used and the absorption was measured at 405 nm after 30 min incubation. In total 21 polyclonal hybridoma clones (HF 1-21) binding to hair samples were identified. However, since shredded hair was coated, both antibodies binding to antigens present inside the hair and to antigens on the hair surface were selected and single cells were cultivated further to generate monoclonals.

To identify binders recognizing structures on the hair surface, antibodies from monoclonal hybridoma lines were tested using the microtiter filtration plate ELISA (see Antibody binding and functionality pp 10). 0.3 mg hair of horse, dog, cat and human were added to separate wells on microtiter filtration plates and incubated in parallel with 100 μl of hybridoma supernatant of each monoclonal to be tested. The test was performed as described above. Visualization of bound antibody was achieved by using a secondary antibody labeled with horseradish peroxidase (HRPO) and 2,2′-azino-bis 3-ethylbenziazoline-6-sulfonic acid (ABTS) reagent as a substrate for HRPO. Colour development was measured at 405 nm after 30 minutes incubation at room temperature. A clear binding specificity of antibody HF 17.6 to horse hair in comparison to dog, cat and human hair can be seen; the low signal is probably due to little antibody in the preparation as the test was performed only two days after selection of the monoclonals and few antibody producing cells existed.

TABLE 2
Binding of monoclonal anti-horse hair antibody
HF 17.6 to hair of four different species.

| | Abs 405 nm | Abs 405 nm | Abs 405 nm | Abs 405 nm |
|---|---|---|---|---|
| | in ELISA on | in ELISA on | in ELISA on | in ELISA on |
| | shredded | shredded | shredded | shredded |
| Clone | HORSE hair | DOG hair | CAT hair | HUMAN hair |
| HF17.6 | 0.081 | 0.007 | 0 | 0 |

ELISA was performed as described above. Binding of antibody HF17.6 to hair of horse, dog, cat and human was evaluated. Values given are the absorbance at 405 nm after 30 min incubation with ABTS with the background subtracted.

### Generation of Cat Hair-Specific Antibodies

Cat hair-specific antibodies were generated through immunization of mice and subsequent use of hybridoma technology. Cut hair from European shorthair cat was shredded using a homogeniser. Shredded hairs were approx. 200 μm in length.

Two mice were immunized with shredded cat hair. Maximal 80 μg hair sample (=20 μL shredded hair) was mixed with 40 μL GERBU adjuvant and 50 μL 1×PBS and used for immunization. Mice were immunized six times at July 1^{st}, July 15^{th}, July 29^{th}, August 12^{th}, August 26^{th} and September 1^{st} and sacrificed at September 3^{rd}. Spleen cells were isolated and fused to myeloma cells to generate hybridomas. Polyclonal hybridoma clones were screened for production of cat-hair-specific antibodies with an ELISA setup as described above. Shredded cat hair in 1×PBS was coated onto low binding 96-well microtiter plates. After addition of the hybridoma cell culture supernatant, binding of specific antibodies was detected using a secondary goat-anti mouse (GAM) antibody conjugated to horseradish peroxidase (HRPO). For detection of antibody binding to hair ABTS-substrate was used and the absorption was measured at 405 nm after 30 min incubation. In total 73 polyclonal hybridoma clones (C2 1-73) binding to hair samples were identified. However, since shredded hair was coated, both antibodies binding to antigens present inside the hair and to antigens on the hair surface were selected. To select antibodies that bind selectively to cat hair, the same type of ELISA was performed 6 days later on human hair. The absorbance given in the following table demonstrates the superior binding to cat hair in comparison to human hair, indicating the production of cat-hair specific antibodies in the polycolonal hybridoma clones.

TABLE 3
Binding of polyclonal anti-cat hair antibodies
to cat or human hair was analysed by ELISA.

| | Abs 405 nm in ELISA on | Abs 405 nm in ELISA on |
|---|---|---|
| Clone | shredded cat hair | shredded human hair |
| C2-2 | 0.89 | 0.26 |
| C2-32 | 0.93 | 0.24 |
| C2-43 | 0.99 | 0.27 |
| C2-45 | 0.92 | 0.22 |
| C2-47 | 1.15 | 0.2 |
| C2-50 | 0.86 | 0.22 |
| C2-68 | 0.93 | 0.2 |

ELISA was performed as described above. Absorption of ABTS was measured after 30 min incubation and no background was subtracted. Abs = absorbance.

### Example

In the following section, an example of an useful embodiment of the invention is provided. It is demonstrated how drug-containing microparticles, attached to dog fur via an antibody which selectively binds to dog hair, are able to protect an animal against pest insects—in this case ticks—for a prolonged period of time and without the side effect of skin irritation.

One common way to apply marketed topical paraciticide formulations to animal hair is to use so called “spot on” formulations in which a small volume of a drug containing solution is deployed onto the neck skin of the animal, having the disadvantage that it often causes severe irritation to the skin. The time period of protection against pest insects is restricted, for instance usually to four weeks in case of ticks. This applies also to other type of formulations with molecular dispersed acaricides.

It is shown that the unique property of the antibody—species selective binding to hair—is maintained when the antibody becomes coupled to a surface of a microparticle. It is demonstrated that the efficacy against ticks (akarizide efficacy >90%) lasts for least three to four weeks. Reduced efficacy >70% can be shown for additional four weeks and can be attributed to the sustained release properties of the particles. Also described is a useful method to link the antibodies to functional groups on the surfaces of the microparticles and a suitable application method to animal fur.

### A) Composition and Production of Drug-Containing Microparticles

The particles are made of polymers. They are non visible on fur, as their size is between 1-10 μm. The active ingredient is encapsulated and released continuously by diffusion. The release can be controlled in a wide range by particle design (polymer type, molecular weight, additives). The particle surface is modified with functional groups, e.g. amine or carboxyl, which are necessary for the linking of the antibody.

In a preferred embodiment, the particles are compounded by the solvent evaporation technique. Therefore the active ingredient, in case of this example the pyrethroid Flumethrin (3-[2-Chloro-2-(4-chlorophenyl)ethenyl]-2,2-dimethylcyclopropanecarboxylic acid cyano(4-fluoro-3-phenoxyphenyl)methyl ester, CAS Nr. 69770-45-2), and the polymer, in this case polystyrene, are dissolved in dichloromethane. The emulsifier, a co-polymer containing polymer segments which are compatible (miscible) with polystyrene and also carboxylic groups COOH which serve as the hydrophilic emulsifying moiety, is dispersed in water. Then the oil phase is dispersed in water with the help of an Ultra Turrax stirrer and the mixture is homogenized with a Microfluidizer. The droplet size is controlled by a microscope. The pH might be set to an alkaline range in order to facilitate the emulsification. The emulsion is then heated up to 60° C. while stirring. At that temperature the dichloromethane evaporates. The emulsion is turned into a suspension, with the free COOH providing a hydrophilic surface. The particle size may be controlled by a mastersizer. The amount of active ingredient is analyzed by HPLC. In case of the given example, the Flumethrin content in the solid polystyrene microparticles is 18% m/m. The formulation code 7c_{—}6+7_EE is given to this type of microparticle.

Microparticles loaded with a fluorescent dye, in this case Uvitex (2,5 thiophenediylbis(5-tert-butyl-1,3-benzoxazole), CAS No. 7128-64-5, 435 nm) are made the same way. The active ingredient is replaced with the dye. The function of these particles is to visualize the antibody mediated bonding of the microparticles to the fur as they are easily detected with a fluorescence microscope.

In another embodiment, the microparticles are loaded by the soaking method. To 10 ml of a 15% dispersion of polystyrene microparticles, modified with surface COOH groups, 2.4 ml of a dichloromethane phase, containing appropriate amounts of Flumethrin, are added. 24 hours of shaking at ambient room temperature follows. The solvent dichloromethane is evaporated afterwards at reduced pressure (150-50 mbar). The dispersion becomes repeatedly centrifuged and the pellets washed with Ethanol/Water mixture to remove unencapsulated and surface bound Flumethrin. After renewed centrifugation, the pellets become redispersed in water and freeze dried. The microparticle encoded BU 163 contained 9% m/m of the drug.

### B) Linkage of the Antibody to the Surface of the Microparticles

The coupling of the hair specific antibody to the drug delivering particle was achieved by the following method:

The carboxylate modified particles are activated by adding a specific amount of N-Ethyl-N′-(3-dimethylaminopropyl)carbodiimide (EDC; 2 mM). Then the activated form is stabilized by the addition of N-Hydroxysuccinimide (NHS; 5 mM). The activated ester complex reacts with primary and secondary amines from the antibodies to give a covalent amide linkage between particle and antibody. In case of the microparticle type 7c_{—}6+7_EE 80 ml of a 2.18% m/V solution were mixed with 29 ml of antibody solution, containing 69.6 mg of a antibody mixture (ratio B7.3/B18-4.3=1:1). BU 163 became equipped with antibodies in a similar way.

The method is described in literature (Hermanson, G. T.: Bioconjugate techniques. Academic press, San Diego, 1996).

### C) Antibody-Mediated Coupling of the Microparticles Specifically to Dog Fur

The proof that the microparticles equipped with the antibodies bind specifically to dog fur is given by spectroscopical detection of the active ingredient. A multi well plate is partly filled with equal amounts (mg) of dog and human hair. The bottom of the wells is a filter with a pore size of 30-40 μm. Those hair containing wells are then filled with a suspension containing the antibody-carrying microparticles 7c_{—}6+7_EE. The multi well plate is then centrifuged in order to remove the liquid and all of the non-bound particles by pressing the supernatant dispersion through the 30-40 μm pores. The particles which are not binding to hair pass the filter as the particle size is only 1-10 μm. Particles binding on hair remain attached to the hair inside the well. The hair is washed five times with a buffer containing 0.05% Tween 20. After each washing, the buffer is again removed by centrifugation. After this, acetonitrile—which dissolves the drug, but not the hair or the material of the multi-well plate—is added to the wells. The acetonitrile extracts the entire encapsulated drug out of the microparticle. The quantitative removal of the active ingredient was proven earlier by HPLC. A transparent multi well plate with no pores at the bottom of the wells is then placed under the hair containing multi well plate. It is made of a polymer which shows only very little UV absorbance. The acetonitrile is centrifuged into the wells of the transparent multi well plates. The active ingredient in the acetonitrile solution can be detected by UV light at 268 nm. After calibration, the UV reader is able to detect the amount of drug present in the well, and therefore indirectly proof the attachment of the microparticles to dog or human hair. The more microparticles are linked to hair the more intense is the UV signal. This correlation is linearly dependent (FIG. 8)

The plot (FIG. 8) proves the binding of considerable amounts of the antibody equipped microparticles to dog hair. The dog hair was taken from seven different breeds (german-shepherd, collie-shepherd, terrier, spitz, schnauzer-labrador, mixed, collie, n=3). In contrast, only a small amount of the microparticles bind to human hair (three different women, untreated undyed hair, n=3), proving the conservation of the species selectivity of the antibodies' active hair binding domains when they become bound to the surfaces of microparticles. It is also demonstrated that the binding strength of the antibody to the animal hair is strong enough to sustain at least five washing cycles with buffer solution.

Instruments: ELISA: Synergy™ HT, BioTek; Microscope: Biozero BZ-8100E, Keyence

### C) Application to Animal Fur as a Spray Formulation

In one useful embodiment of a formulation, the antibody-equipped drug-containing microparticles are applied to the animal fur as a spray formulation.

In the given example, 6 mg of encapsulated Flumethrin per kg bodyweight are applied to the dog. A beagle dog weights 11 kg on average. Therefore 367 mg of the anti-body equipped microparticles 7c_{—}6+7_EE containing 66 mg of Flumethrin—as the drug concentration in the microparticles is 18% m/m—are applied to the animals. Depending on the active ingredient concentration of the particles and the amount of the drug needed, this value will vary. For the microparticles BU163 733 mg are used, as the drug content is 9% m/m. 100 mg of the fluorescent particles and the calculated amount of the drug-loaded microparticles are dispersed in a suitable amount of water. In case of this example, 30 ml of water are used. A small amount of surfactant (in this example 0.01% Tween 20) is added to facilitate the spreading of the formulations on the sometimes fatty dog hair. The dispersions are filled into a common bottle, equipped with a pump spray head which is suitable to spray particle suspensions without blocking the valve, and are ready to use. The dispersions are sprayed evenly all over the dog fur, allowing a homogenous distribution of the microparticles onto the dog hair.

E) In-Vivo Study: Efficacy Against Ticks (Rhipicephalus sanguineus) on Dogs

The objective of this study was to evaluate the effectiveness against ticks of two different spray formulations containing flumethrin loaded microcarriers bound to dog fur via antibodies on dogs experimentally infested with Rhipicephalus sanguineus (table 4).

Nine dogs were enrolled in this study in three groups of three dogs each. Each dog was housed in individual cages for the whole study period.

On study day (SD) −1 all dogs were sedated approximately 0.1 ml/kg BW Ketaminhydrochlorid (Ketamin®10%) in combination with approximately 0.1 ml/kg BW Xylazinhydrochlorid (Xylazin® 2%) i.m. During the sedation all dogs were laid in sternal or lateral recumbency on the floor and 50 Rhipicephalus sanguineus ticks (25♀, 25♂) were released onto the back of the dogs. The dogs were sleeping for 1 h to 1.5 hours and therefore restrained from removing the ticks before they were able to attach.

On SD 0 tick infestation rate was determined without removal of the ticks by intensive adspection/palpation of the total body surface: Head, ears, neck, lateral areas, dorsal strip from shoulder blades to base of tail, tail and anal area, fore legs and shoulders, hind legs, abdominal area from chest to inside hind legs, feet. All live attached female ticks were counted and the dogs were randomly allocated to three study groups based on the tick counts.

After tick counting on day 0 treatment was performed. Dogs of the control group were left untreated and served as negative control group. The dogs of the two treatment groups were dosed once with the IVP (Investigational Veterinary Product). Each IVP had a volume of 30 ml and contained 66 mg flumethrin as active ingredient. Each dog received the total amount of 30 ml irrespective of the body weight. With the dog standing each dog was sprayed with the IVP evenly over the whole body.

TABLE 4
Overview of the microparticle formulations used in the
in-vivo experiment, and information about dosing

| | | Dosage | Total volume |
|---|---|---|---|
| Group | IVP | [mg/kg BW] | applied |
| 1 | 7c_6 + 7_EE | 6 mg/kgBW; | 30 ml |
| 2 | BU 163 | average of dog | |
| | | weight 11 kg | |
| | | →66 mg/dog | |
| 3 | Control | n.a. | n.a. |

n.a. not applicable

On the day of the treatment (Study Day SD 0) the dogs were observed for adverse events at two and four hours post treatment. All dogs tolerated the treatment well. On SD 2 tick counts were performed in the same way as on SD 0 but with removal of the ticks. Ticks were identified as free or attached, engorged or unengorged, live or dead ticks. In regular intervals each dog was reinfested in the same manner and tick counting after 48 hours was performed as described. The chosen intervals were one, three, seven and nine weeks after treatment, e.g. tick infestation was done on SD 5, 20, 48 and 61, and tick counting two days later on SD 7, 22, 50 and 63.

Besides at each tick counting day (48 hours after infestation) 100 mg hair of each dog was taken in regular intervals. The fur samples were taken from all over the dog: shoulder, back, from the beginning of the tail, hip and investigated by fluorescence microscopy. Hair samples were taken at Days 2, 7, 14, 42 and 63 after the administration of the formulation.

Detailed general health observations were performed on all dogs during tick counting procedures. Special attention was paid to skin irritations. During the whole study period no skin irritations due to the treatment could be observed.

The tick counts were used to evaluate the efficacy of the IVP. Percent efficacy was calculated with a modified Abbott formula according to the recommendations for controlled tests described in the guideline EMEA/CVMP/005/00-Final:

% Efficacy=(N2−N1)/N2×100

N1=Geometric mean tick count for the group treated with IVP
N2=Geometric mean tick count for the control group

“Tick count” is defined as “ticks representing a treatment failure”. In case of determination of curative efficacy (SD 2) only live ticks were considered as treatment failure, whereas in case of determination of preventive efficacy (all remaining counting time points) live ticks and dead engorged ticks were considered as treatment failure.

An active ingredient is seen as highly effective if an efficacy of >90% is achieved 48 h after treatment and weekly reinfestation. Efficacy should be given over a three to four week period.

TABLE 5
Calculated efficacy [%]

| group | SD 2 | SD 7 | SD 22 | SD 50 | SD 63 |
|---|---|---|---|---|---|
| Treatment group 1 | 97.2 | 100 | 97.4 | 78.3 | 56.2 |
| Treatment group 2 | 63.4 | 94 | 97.4 | 85.2 | 29.4 |

The minimum goal to show efficacy over 90% a period of three to four weeks was achieved (table 5). Given the high concentration of flumethrin in the particles, this form of application clearly protects the animals from skin irritation.

After 50 days post treatment reduced efficacy still is notable. The release of flumethrin from the microparticles after the study period of ≧50 days may not be sufficient to keep active levels of flumethrin on the skin of the animals. The better protection during the first weeks of the trial may be attributed to a certain part of free flumethrin and a higher diffusion rate from the particles in the early phase due to the high flumethrin concentration inside the matrix.

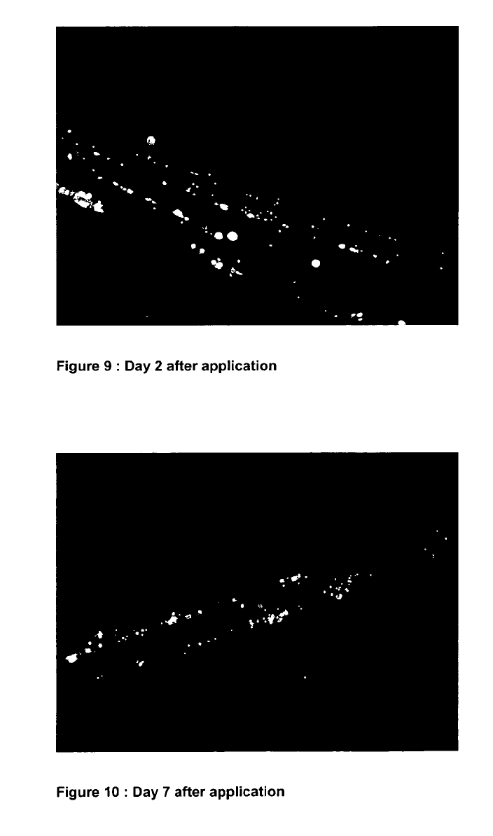

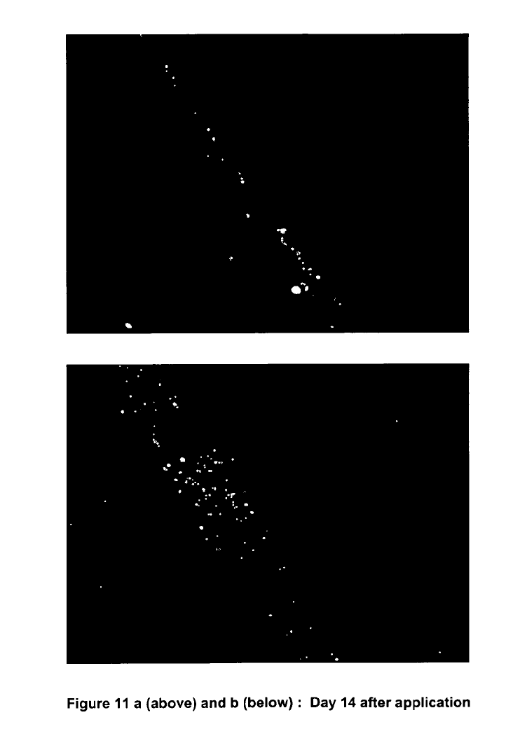

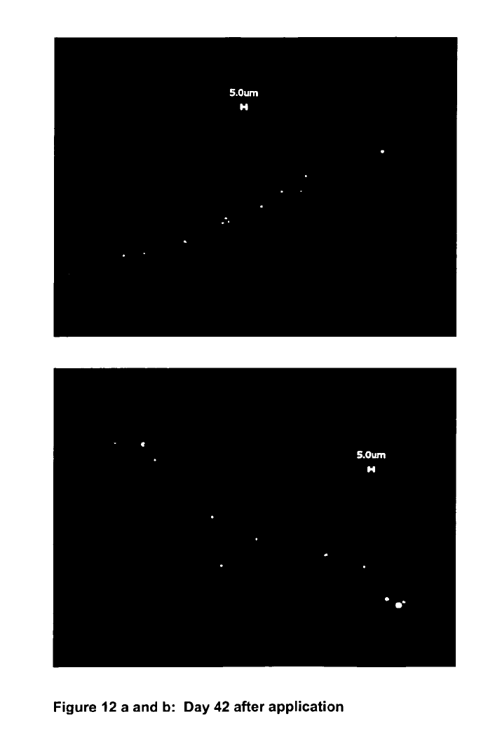

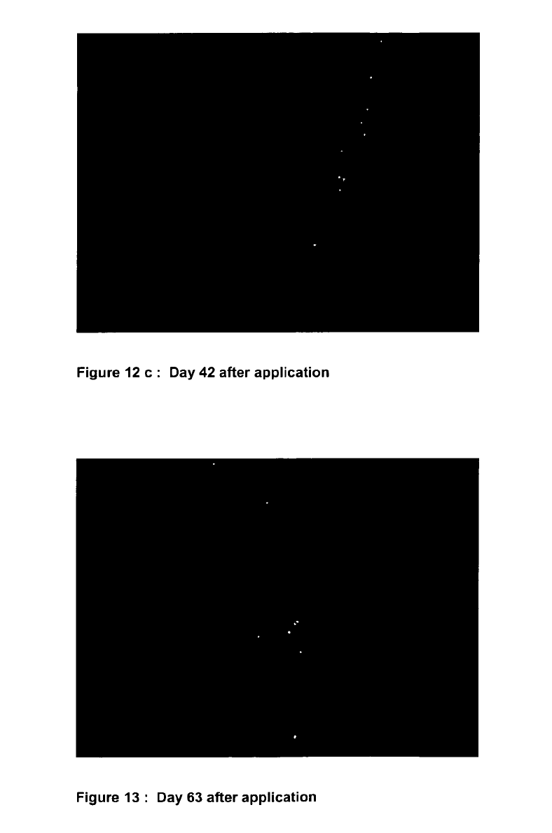

By fluorescence imaging, the persistent attachment of the microparticles to the dog fur can be detected. It can be shown that microparticles remain attached to the hair via antibody binding for the whole period of the experiment (FIGS. 9 to 13). Note that the fluorescence-dye marked particles serve as indicator only and represent only 20% of the total number of microparticles in the formulations. With time, less particles can be detected, partially because they have fallen off, partially because the fluorescent dye bleaches out because of the constant exposure to daylight.

F) Conclusions from the Example

The described example demonstrates one highly useful embodiment of the presented invention: The equipment of drug-containing microparticles which are suitable to control the release of the drugs over a wide range, with antibodies which specifically bind to animal hair (in this case dog hair). There are multiple advantages of this application of the invention:
It allows to target paraciticides to a specific mammal species, thus considerable reducing the danger of cross-application or drug transfer, to e.g. humans or other mammal species, when the formulations are applied or when the different species interact, for instance by playing or licking.
It allows to direct the drug to animal fur, therefore avoiding largely contact with the skin which reduces the potential for skin irritation or severe skin damage
The universality of the principle of linking a single species-specific antibody to different microparticle surfaces via COON, NH2 or other groups and deploy the microparticles to fur, allows to select a range of different microparticle types (different polymers containing different drugs with different release profiles) for a single formulation. It permits combinations of different type of microparticles in a dispersion and therefore to combine drugs in a single formulation which may not be chemically compatible otherwise.
The use of suitable microparticles with appropriate drug release profiles allows to extent the efficacy of the drug to several weeks or months, depending on the particle and drug type, therefore providing the potential of considerably extending the efficacy period of common topically applied liquid pesticide and other drug formulations.
The attachment of the microparticles to the animal hair is strong enough to overcome intense contact with water or even mild washing, e.g. when the animal is strolling around in rainy weather or is swimming in a pond for some time. In this case, regular formulations require often renewed application.

1. An antibody which selective binds to the surface of hair of animals.

2. The antibody according to claim 1 selected from the group comprising polyclonal antibodies, monoclonal antibodies or recombinant antibodies such as full-size antibody, dimeric secretory IgA, multimeric IgM and fragments thereof such as F(ab′)2-fragment, Fab-fragment, Fv-fragment, single chain Fv antibody (scFv), bispecific scFv, diabody, triabody, tetrabody, dingle domain antibody (dAb), minobody or molecular recognition unit (MRU).

3. The antibody according to claim 2 which is derived from hybridoma cells, synthetic, semi-synthetic, naïve and immunocompetent phage libraries or ribosome display libraries, or by the generation of fully synthetic designer antibodies.

4. The antibody according to claim 1 selectively binding to the hair of one animal species.

5. The antibody according to claim 1 selectively binding to the hair of dog, cat, cattle, sheep, goat, camel, llama and/or horse.

6. A drug delivery system comprising the antibody according to claim 1.

7. A drug delivery system comprising the antibody of claim 1 which is in addition bound or attached to the surface of solid particles, thus selectively binding these particles to animal hair.

8. A drug delivery system according to claim 7 comprising of a formulation of the antibodies selectively binding to animal hair and particles containing an active ingredient, in such a way that the antibody is binding selectively to animal hair in a first step and the particles are bound to the antibody coupled to the animal hair in a second step.

9. A drug delivery system according to claim 7 comprising a formulation of the animal hair binding antibody linked or chemically bound to a suitable spacer/mediator/intermediate coupling agent and of particles, in such a way that the spacer/mediator/coupling agent carrying the antibody becomes selectively bound to animal hair via said antibody in a first step and that the particles become linked to the animal hair by binding to said spacer/mediator/coupling agent in a second step.

10. A drug delivery system according to claim 7 in which the particles contain active ingredients or agents beneficial for the animal, namely drugs, insecticides, hair or skin caring agents, repellent agents which protect the treated animal from being attacked by harmful pest animals, e.g. insects, smell modifying agents or agents modifying the behavior of the treated animal or other non-treated individual animals which interact with the animal to which the treatment is applied.

11. A drug delivery system of claim 4 where the particles contain active ingredients or agents beneficial for the animal in a concentration suitable for its therapeutic or beneficial purpose, namely 0.01% to 99.9%.

12. A drug delivery system of claim 4 where the particles have a diameter of 0.001 μm to 10 μm, preferably 0.1 μm to 2 μm.

13. A drug delivery system of claim 4 where the microparticles contain a drug of the pyrethoid type which is administered to the fur of animals and delivers the pyrethoid over a prolonged period of time and without causing side effects, namely skin irritation.
